(19) United States
(12) Patent Application Publication
(10) Pub. No.: US 2008/0249627 A1
(43) Pub. Date: Oct. 9, 2008

(54) Prosthetic Disc Device and Method for Intervertebral Disc Replacement

(75) Inventors: Jeff W. Moehlenbruck, Austin, TX (US); Steven L. Griffith, Eden Prairie, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 11/697,754

(22) Filed: Apr. 9, 2007

(51) Int. Cl. A61F 2/44

(52) U.S. Cl. 623/17.16; 623/17.13

(57) ABSTRACT

An implant and method for insertion between two adjacent vertebrae includes an implant body having a leading end and a trailing end spaced apart by a longitudinal dimension of the implant, two diametrically opposed first and second shells, each of the shells having a body portion and a thread portion extending outwardly from the body portion, and a resilient support portion disposed between the two shells. The support portion may be made of an elastomeric material or may include a spring mechanism.

### FIELD OF THE INVENTION

The present invention generally relates to intervertebral implants and, more particularly, to threaded intervertebral implants in the human spinal column.

### BACKGROUND OF THE INVENTION

The human spinal column is formed from 24 vertebrae, which are separated by and coupled to each other at their axial surfaces by intervertebral discs. Intervertebral discs are soft and pliable cartilaginous cushions interposed between the vertebrae. The discs resist compression along the axis of the spinal column while simultaneously permitting constrained flexion, extension, and rotation between the vertebrae. When working together—like links in a bicycle chain—the discs serve to provide the characteristic smooth motion and flexibility of the healthy spine. The quiet but critically important intervertebral discs are, however, markedly vulnerable to injury and disease.

This vulnerability of the intervertebral discs is, in part, a consequence of the high compressive forces to which this cartilaginous tissue is usually subjected. Because blood vessels cannot remain open and function under high compressive loads, the intervertebral discs constitute the largest avascular structures in the body. Avascular disc tissues are nearly incapable of self-repair in response to damage, insult, or injury. The degenerative process that ensues after injury may take a very long time, sometimes months to years, to progress after the original traumatic event or underlying cause. Given the active lifestyles of the general populace, this intrinsic vulnerability provides an explanation for the observation that back pain (with corresponding degenerative disc disease) is one of the most common medical ailments in the Western world. There are millions of adults in the United States who suffer from chronic low back or neck pain, with the primary culprit believed to be intervertebral disc degeneration.

Surgical treatments for intervertebral disc herniation and chronic intervertebral disc degeneration include discectomy (removal of protruding or herniated disc tissues) and/or spinal fusion (complete disc removal and bone fusion of the two adjacent vertebrae). Thousands of discectomies and spinal fusions are performed in the U.S. each year to treat herniations and disc disease.

Many devices and methods have been developed which provide for the removal of damaged or degenerated intervertebral discs and subsequent bony interbody fusion of the vertebrae. These devices and methods are designed to restore lost intervertebral height and to provide permanent stabilization of the spine. Fusion surgery may require more donor bone than is available locally at the primary surgical site. An additional surgical procedure may be performed at that time, usually in the hip, for the harvest of additional bone. Autogenous grafts of dowel-shaped sections of bone, harvested from the iliac crest of the hip, may be implanted between the vertebrae to distract and allow bone growth across the intervertebral space. Thus, this procedure creates a fusion of the adjacent vertebrae into one bone mass.

Another alternative of vertebral stabilization involves the implantation between adjacent vertebrae of a perforated cylindrical cage, such as the BAK™ Interbody Fusion device (“BAK™ Cage”) commercially available from Zimmer Spine, Inc. (the assignee of the present invention). Bone fragments produced in preparing the vertebrae for the implantations as well as autogenous bone harvested from the patient's hip during the surgery are inserted into a cage to promote bone growth and eventual fusion around and through the cage.

Vertebral stabilization by fusion of adjacent vertebrae has proven successful in permanently preserving intervertebral spacing and resolving back pain symptoms while reducing some of the spine's normal range of motion, and thereby reducing the subject's spinal flexibility. Other devices have been developed to restore disc height, thereby stabilizing the spine segment, while retaining a certain amount of the natural motion of the affected spine segment. These devices are designed to replace a diseased intervertebral disc with a prosthesis that is “jointed” to permit relative movement between vertebrae. Fixation of the prosthesis and the vertebrae, however, may be relatively complex.

The intended movement between the components of earlier jointed prostheses can cause relative motion between the prosthesis and adjacent bone surface(s). Because such motion may limit bone ingrowth and stability of the interface, disc prostheses have been designed for greater compatibility with attachment by bone ingrowth. In addition, because the joint elements of these devices typically may need to occupy a substantial vertical extent in order to achieve the desired range of motion while fitting within the intervertebral space, attachment of such devices has been generally effected by use of flat plates or surfaces provided on either side of the joint elements as points of fixation to the vertebrae.

Attachment may be accomplished by compressive or friction fits, spiked projections, screws or pins, complemented in some instances with tissue ingrowth into porous surfaces. Moreover, several such devices may use attachment flanges that extend beyond the surfaces of the vertebrae to which the device is attached, which may add to the complexity of the surgical procedure. In addition, implantation of and subsequent revisionary surgical procedures involving such devices may require anterior access to the spine, which may be complex.

It would therefore be desirable to have a spinal implant effective in permanently maintaining intervertebral spacing to prevent nerve or spinal cord compression while preserving as much of the natural range of motion between the affected vertebrae as possible. It would be further desired for such a device to be capable of forming a permanent, strong attachment to the vertebrae while not protruding beyond the external surfaces to which it is attached.

It would also be desirable to have a method of replacing a damaged or displaced disc that maintains intervertebral spacing to prevent nerve and spinal cord compression, while preserving the natural relative motion between the vertebrae. It would further be desirable for such method to be less complex than known methods of carrying out such replacement.

### SUMMARY OF THE INVENTION

The present invention overcomes the foregoing and other shortcomings and drawbacks of threaded intervertebral implants in the human spinal column heretofore known. While the invention will be described in connection with certain embodiments, it will be understood that the invention is not limited to these embodiments. On the contrary, the invention includes all alternatives, modifications and equivalents as may be included within the spirit and scope of the present invention.

In accordance with one embodiment of the invention, an implant for insertion between two adjacent vertebrae includes an implant body having a leading end and a trailing end spaced apart by a longitudinal dimension of the implant, and two diametrically opposed shells. Each of the shells has a body portion and a thread portion extending outwardly from the body portion. The implant includes a resilient support portion disposed between the two shells. Each thread portion may further extend substantially between the ends. The support portion may be adapted to collapse in a direction generally orthogonal to the longitudinal dimension, thereby permitting the two shells to jointly define a threaded cylindrical body. The support portion may further be adapted to maintain the shells spaced apart.

In another embodiment, the first and second shells may be made of metal, while the support portion may be made of an elastomeric material or may include a spring mechanism.

In another embodiment, the two diametrically opposed first and second shells and the resilient support portion are integrally formed and are generally made from a material having at least two distinct regions, each region respectively having first and second sets of physical properties, whereby at least a portion of the shells is made from material of the first region and the support portion is made from material of the second region.

In another embodiment, the support portion is adapted to collapse in a direction generally parallel to the longitudinal dimension of the implant body, thereby increasing a spacing between the first and second shells. The device may include an actuator. Such actuator may, for example, include a threaded elongate member and a fastener adapted to threadably receive the elongate member.

In another aspect of an embodiment, the first and second shells may be shaped to conform to opposed surfaces of adjacent vertebrae. The first and second shells may further conform to opposed surfaces of adjacent vertebrae oriented at an angle from one another, such as vertebrae in a lordotic spine segment.

In yet another embodiment, a method of restoring an intervertebral disc height between two adjacent vertebrae includes posteriorly accessing a spinal column segment defined by the two vertebrae and inserting at least one implant between the two vertebrae. The implant may have two opposed body portions and a resilient support portion disposed between the two body portions.

The method may include defining an intervertebral bore between the two adjacent vertebrae prior to inserting the implant and may further include bringing the two body portions toward each other prior to such insertion. The method may also include threadably engaging the implant with each of two confronting vertebral surfaces defining the intervertebral disc height, which may further include threadably engaging the implant with the cortical rim defining each of the two surfaces. The method may also include inserting two implants between the two vertebrae.

Advantageously, the embodiments of the device herein described restore intervertebral disc height that has been lost as a consequence of degenerative disc disease or spondylolisthesis while avoiding a bony bridging fusion between two adjacent vertebrae. The device is instead designed to preserve mobility in a spinal motion segment.

Moreover, the methods herein described permit posterior implantation of a prosthetic disc device aimed at restoring intervertebral disc height while preserving mobility in a spinal motion segment.

The above and other objects and advantages of the present invention shall be made apparent from the accompanying drawings and the description thereof.

### BRIEF DESCRIPTION OF THE DRAWINGS

These and other objectives and advantages will become readily apparent to those of ordinary skill in the art from the following description of embodiments of the invention and from the drawings in which:

FIG. 1 is a perspective view of a prosthetic disc device in accordance with one embodiment of the invention.

FIG. 2 is a cross-sectional view taken along line 2-2 of FIG. 1.

FIG. 3 is a perspective view of another embodiment of a prosthetic disc device.

FIG. 4 is a cross-sectional view taken along line 4-4 of FIG. 3.

FIG. 5 is a cross-sectional view of an alternative embodiment of a prosthetic disc device including a spring mechanism defining the core element.

FIG. 6 is a perspective view of the prosthetic disc device of FIG. 1 in a compressed, pre-deployment state.

FIG. 6A is a perspective view of the prosthetic device of FIG. 1 prior to insertion into an intervertebral bore.

FIG. 6B is a perspective view of two of the prosthetic disc devices of FIG. 1 deployed between two adjacent vertebrae.

FIG. 7 is a perspective view of another embodiment of a prosthetic disc device.

FIG. 8A is a cross-sectional view taken along line 8A-8A of FIG. 7.

FIG. 8B is a cross-sectional view similar to FIG. 8A showing a core element of the prosthetic disc device of FIGS. 7, 8A in a compressed state.

FIG. 9 is a side view of an embodiment of a prosthetic disc device implanted in an intervertebral space.

FIG. 10 is a side view of another embodiment of a prosthetic disc device implanted in an intervertebral space in a lordotic segment of a spine.

### DETAILED DESCRIPTION OF THE PREFERRED EMBODIMENTS

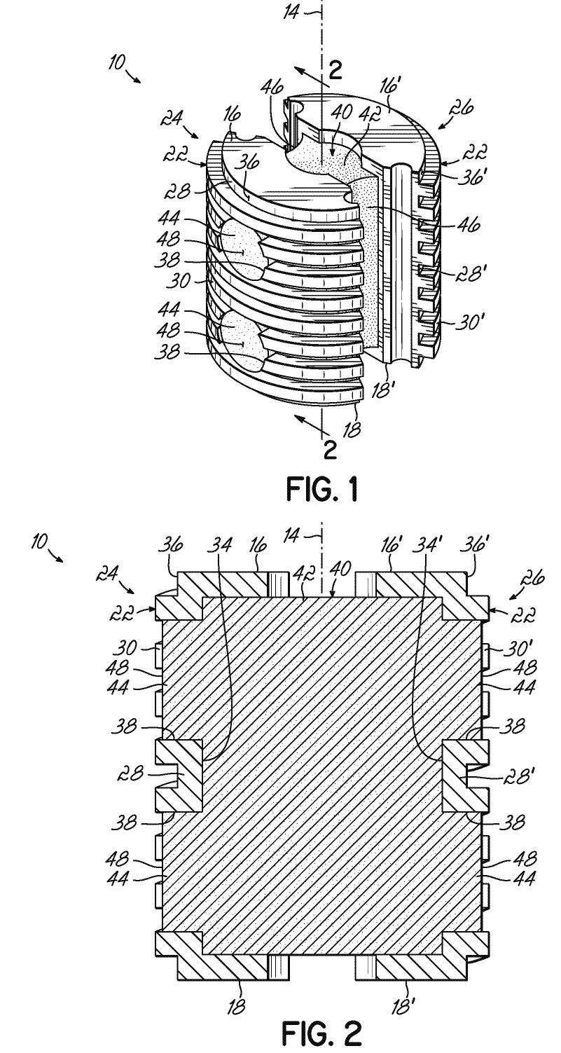

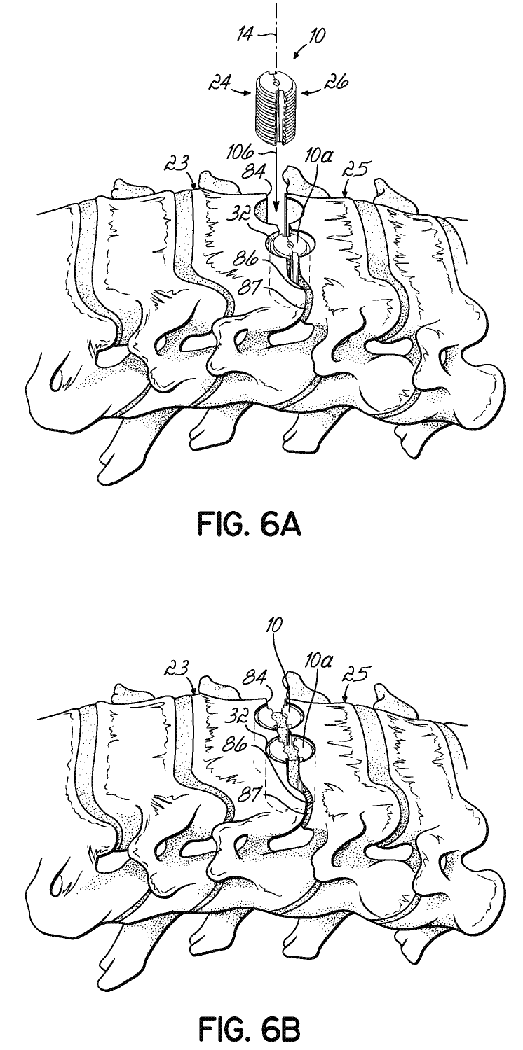

With reference to the figures and more particularly to FIGS. 1-2, an intervertebral prosthetic disc device 10 includes a central axis 14, two spaced and opposed first and second shells 24, 26, each respectively having an body portion 28, 28′, a thread portion 30, 30′ extending outwardly therefrom, proximal end portions 16, 16′ and distal end portions 18, 18′ such that the prosthetic disc device 10 can be threadably engaged with a suitable surface such as the surfaces defining an intervertebral bore 32 (FIG. 6A) when both shells 24, 26 are brought together. Each of the inner body portions 28, 28′ includes an inner surface 34, 34′ and an outer surface 36, 36′. In one embodiment, the shells 24, 26 may be formed from the upper and lower halves of a threaded, cylindrical, hollow cage apparatus that has been split along the central axis, such as the BAK™ cage device commercially available from Zimmer Spine, Inc., the assignee of the present invention. While the embodiment of FIGS. 1-2 depict proximal end portions 16, 16′ and distal end portions 18, 18′ having respective continuous surfaces, persons of ordinary skill in the art will appreciate that, alternatively, the shells 24, 26 may include body ends of a shape and surface different from that shown, including, for example, surface discontinuities or may be such that the proximal end portion16, 16′ is shaped differently from the distal end portion 18, 18′. Alternatively, shells 24, 26 may include only one end portion or include no end portions at all.

With continued reference to FIGS. 1-2, the surface of each of the two shells 24, 26 of prosthetic disc device 10 includes generally bores 38 providing discontinuity of the body portions 28, 28′ and discontinuity of the thread portions 30, 30′. Although the bores 38 are depicted in a number of two per shell 24, 26, persons of ordinary skill in the art will appreciate that, alternatively, each shell 24, 26 may include any suitable number of bores 38 of any suitable shape (other than the depicted exemplary round shape of bores 38) and dimensions including but not limited to bores of relatively different shapes on each shell 24, 26. Moreover, while the embodiment of FIG. 1 depicts bores 38 aligned along an axis generally parallel to the central axis 14 with each bore 38 extending perpendicular to the axis 14, any arrangement or configuration of any number of bores 38 is contemplated, so long as the bores 38 do not substantially interfere with the structural integrity of the prosthetic disc device 10. Alternatively, shells 24, 26 may include no bores 38 at all.

The thread portions 30, 30′ of prosthetic disc device 10 in the exemplary embodiment of FIGS. 1-2 are spirally wound respectively around the body portions 28, 28′. The thread portions 30, 30′ are configured such that they can engage opposing surfaces of adjacent vertebrae 23, 25 (FIG. 6A) to draw the prosthetic disc device 10 in the direction of the axis 14 upon rotation of the prosthetic disc device 10 about the axis 14. While single threading is shown defining each thread portion 30, 30′, persons of ordinary skill in the art will appreciate that, alternatively, double threading or multiple threading in excess of double threading could be used. The thread portions 30, 30′ in this embodiment have a generally rectangular profile but they may alternatively have any other suitable shapes such as triangular, polygonal or irregular or further include sharp ends, so long as such profiles are suitably chosen to engage bone surfaces such as those defining the intervertebral bore 32 (FIG. 6A).

With continued reference to FIGS. 1-2, the body portions 28, 28′ and thread portions 30, 30′ are formed from a biocompatible and rigid material such as ceramic, titanium, steel or a composite alloy or any other material suitable for implantation in the human body. Titanium and titanium alloys may, for example, be chosen because they are non-corrosive and fatigue resistant and because they are widely used in prosthetic disc devices. Moreover, the body portions 28, 28′ and thread portions 30, 30′ may be made of the same material or, alternatively, be made of different materials. The body portions 28, 28′ may, for example, be made of one material while the thread portions 30, 30′ may be made of another. Likewise, materials defining a first body portion 28 of a prosthetic device 10 may be different from the materials defining an opposed second body portion within the same prosthetic disc device 10.

While the depicted exemplary embodiment includes arcuate outer surfaces 36, 36′ respectively defining each of the body portions 28, 28′, persons of ordinary skill in the art will appreciate that, alternatively, outer surfaces 36, 36′ may have any other shape or shapes suitable to support thread portions 30, 30′. Moreover, although the thread portions 30, 30′ are depicted as substantially continuous (but for the discontinuities provided by the bores 38) and extending substantially between the proximal and distal ends 16, 18, discrete thread portion segments positioned throughout the surfaces of each shell 24, 26 are contemplated extending substantially between the proximal portions 16, 16′ and distal end portions 18, 18′ or anywhere in between.

With continued reference to FIGS. 1-2, the prosthetic disc device 10 includes an irregularly shaped core element 40 providing a resilient spacing between the two opposed first and second shells 24, 26. Core element 40 includes a central portion 42 and distal portions 44. The central portion 42 may be irregularly or regularly shaped or be formed, as depicted in the illustrative embodiment of FIG. 1, having two opposed lateral arcuate surfaces 46 on each side of central axis 14. The distal portions 44 of the core element 40 are irregularly shaped and substantially fill the volumes defined by the inner surfaces 34, 34′ of the body portions 28, 28′ and through the bores 38. While the illustrative embodiment of FIGS. 1-2 depicts distal portions 44 having round end surfaces 48 defined by segments protruding through the bores 38, distal portions 44 may alternatively further extend over some of the outer surfaces 36, 36′ of the body portions 28, 28′.

The distal portions 44 of the core element 40 are suitably engaged to some or all of the inner surfaces 34, 34′, outer surfaces 36, 36′, threaded portions 30, 30′ and surfaces defining the bores 38. Engagement of the distal portions 44 may include mechanical entanglement, adhesive bonding, thermal bonding, chemical bonding or any other suitable method or components.

With reference to FIGS. 1-2, 6A, the core element 40 includes a suitable material or combination of materials capable of providing relative motion between two adjacent vertebrae 23, 25, maintaining integral unity of the prosthetic disc device 10 and providing compressive resistance against a force exerted by the two adjacent vertebrae 23, 25. The core element 40 may, for example, be made of a flexible, elastomeric polymer material such as polyurethane or silicone rubber.

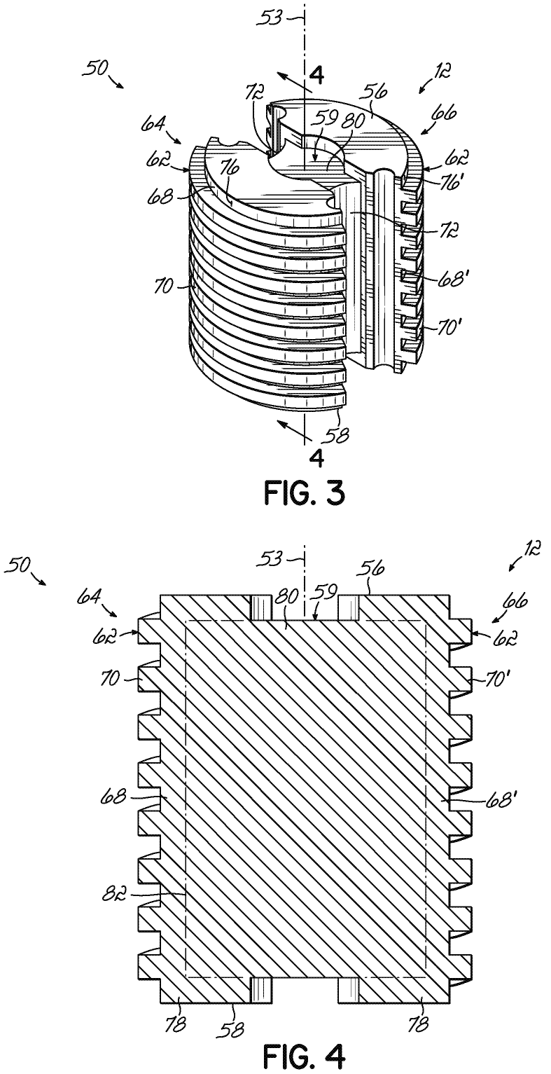

With reference to FIGS. 3-4, in which like reference numerals refer to like features in FIGS. 1-2, an alternative embodiment of a prosthetic disc device 50 includes a central axis 53, a proximal end 56 and a distal end 58. Prosthetic disc device 50 also includes a thread portion 62 generally extending between the proximal and distal ends 56, 58, and a core portion 59. The body portion 60, thread portion 62 and core portion 59 are all integrally formed with each other.

The prosthetic disc device 50 is further defined by two spaced and opposed shells 64, 66 each respectively having a body portion 68, 68′ and a thread portion 70, 70′ extending outwardly therefrom such that prosthetic disc device 50 can be threadably engaged with a surface such as the surfaces defining intervertebral bore 32 (FIG. 6A).

With continued reference to FIGS. 3-4, the core portion 59 extends along the central axis 53 substantially between the ends 56, 58 and interconnects the shells 64, 66 to maintain the unitary integrity of the prosthetic disc device 50. The core portion 59 may be irregularly or regularly shaped or be formed, as depicted in the illustrative embodiment of FIG. 3, having two opposed lateral arcuate surfaces 72 each on one side of central axis 53.

The prosthetic disc device 50 is defined by a material fabricated such that the device has different properties throughout its volume. In the exemplary embodiment of FIGS. 3-4, the material defining the device 50 includes two continuously formed first and second regions 78, 80 respectively bounded by a transition area represented by line 82. The first region 78 generally defines the body portions 68, 68′, the thread portions 70, 70′ and ends 56, 58, while the second region 80 defines the core portion 59. Alternatively, the first and second regions may be bounded anywhere throughout the volume defined by the prosthetic disc device 50. Alternatively also, prosthetic disc device 50 may be formed of a material having more than two regions.

In the exemplary embodiment of FIGS. 3-4, the material properties in the first region are such that the body portions 68, 68′ and the thread portions 70, 70′ are generally rigid, thereby permitting threaded engagement of the prosthetic disc device 50 with bone surfaces such as those defining intervertebral bore 32 (FIG. 6A). The material properties in the second region 80 are such that the core portion 59 is capable of providing relative motion between two adjacent vertebrae 23, 25 (FIG. 6A), maintaining integral unity of the prosthetic disc device 50 and providing compressive resistance against a force exerted by the two adjacent vertebrae 23, 25. Moreover, the transition between the first and second regions 78, 80 may be abrupt or gradual. A gradual transition may be desirable, for example, to minimize the probability of stress concentrations. Persons of ordinary skill in the art will appreciate that all alternative variations described in regard to the prosthetic disc device 10 of FIGS. 1-2 are applicable to prosthetic disc device 50 as well.

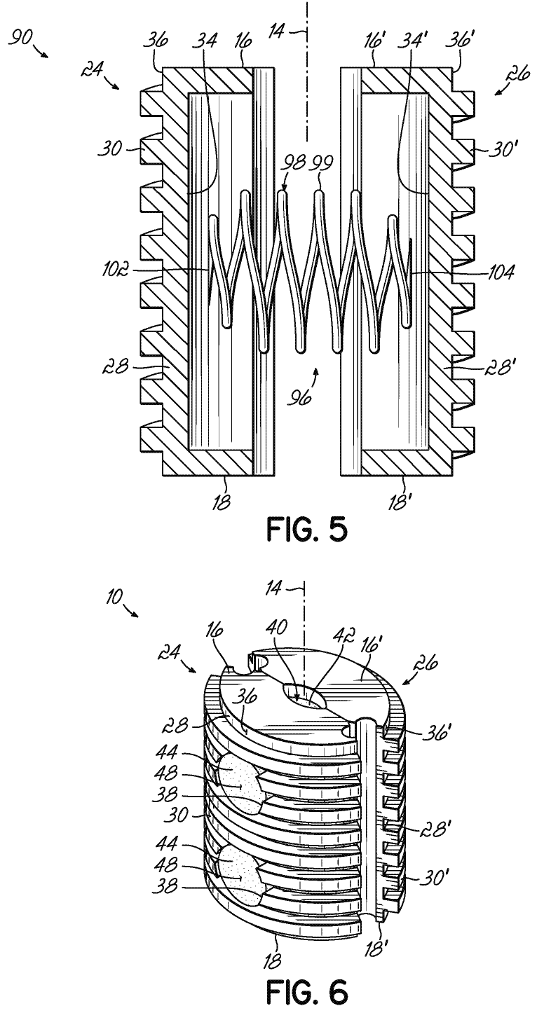

With reference to FIG. 5, in which like reference numerals refer to like features in FIGS. 1-2, an alternative embodiment of a prosthetic disc device 90 is similar in most respects to the prosthetic disc device 10 of FIGS. 1-2, the description of which may be referred to for an understanding of prosthetic disc device 90 as well. Prosthetic disc device 90 includes two diametrically opposed shells 24, 26 and a core element 96 defined by a spring mechanism. In the exemplary embodiment of FIG. 5, the spring mechanism is defined by a compression spring 98 made from a wire 99 and having ends 102, 104 each suitably coupled to each of the shells 24, 26 such that it provides unitary integrity to the prosthetic disc device 90 and relative motion of each of the shells 24, 26 with respect to the spring 98. Such coupling may, for example, include mechanical engagement with one or more of the surfaces of the thread portions 30, 30′, body portions 28, 28′ or bores (similar to bores 38 of FIG. 1), if present. Persons of ordinary skill in the art will appreciate that all alternative variations described in regard to the prosthetic disc device 10 of FIGS. 1-2 are applicable to prosthetic disc device 90 as well.

The spring 98 is made of a suitable material and suitable design to enable temporary joining of the two shells 24, 26 to permit deployment of prosthetic disc device 90, as depicted in FIG. 6A. The spring 98 is further defined by a material and design such it can exert a force against two adjacent vertebrae such as vertebrae 23, 25 (FIG. 6A) thereby creating a space therebetween. The spring 98 may, for example, be made of steel, titanium, a titanium alloy or other suitable materials, including but not limited to metals, suitable for implantation in a human body. While the embodiment of FIG. 5 is depicted having a spring mechanism defined by a compression spring 98 made of a single wire 99, persons of ordinary skill in the art will appreciate that, alternatively, the spring mechanism may include other spring designs defined by one or more other wire or non-wire structures.

With reference to FIGS. 6-6A, the exemplary prosthetic disc device 10 (best described in FIGS. 1-2) is depicted in a compressed, pre-deployment state prior to insertion and threadable engagement with the surfaces defining an intervertebral bore 32. Insertion follows the general direction of arrow 106, substantially in the direction of the central axis 14 of the device 10, until a desired point in the intervertebral bore 32 is reached. In this pre-deployment state, the prosthetic disc device 10 is depicted compressed, with the core element 40 collapsed such that the two shells 24, 26 jointly define an externally threaded cylindrically-shaped prosthetic disc device 10. This compressed state may be maintained via the use of a suitable tool or, alternatively, via locking elements (not shown) holding the two shells 24, 26 together.

With reference to FIG. 6B, in its deployed position, prosthetic disc device 10 is shown next to another exemplary prosthetic disc device 10a spaced therefrom. The prosthetic disc device 10 is depicted situated such that the central axis 14 is generally parallel to each of the planes 84, 86 respectively defined by the opposed surfaces of two adjacent vertebrae 23, 25. The prosthetic disc device 10 is further shown oriented such that the first and second shells 24, 26 are oppositely located along an axis orthogonal to the planes 84, 86. In this configuration, the first and second shells 24, 26 exert respective forces against the two opposed surfaces of the vertebrae 23, 25, thereby providing a space 87 between them. In another aspect of the embodiment depicted in FIG. 6B, the prosthetic disc device 10 may further contact the cortical rim defining the surfaces of each of the two opposed surfaces of the vertebrae 23, 25, such that the device 10 is securely held within the intervertebral space. In the deployed state depicted in FIG. 6B, furthermore, bone ingrowth may occur such that bone matter from vertebrae 23 can extend into areas between adjacent thread segments defining thread portions 30, 30′ (FIG. 1) not filled by the distal portions 44 (FIG. 1) of the core element 40. Such bone ingrowth causes fixation of each of the shells 24, 26 with a respective vertebra 23, 25, thereby preventing slippage or rotational motion of a shell 24, 26 with respect to the surface of the vertebra 23, 25. Once the desired position in the intervertebral bore 32 is reached, the tool or locking elements (not shown) is/are disengaged such that the core element 40 is allowed to expand, causing the two shells 24, 26 to push away from each other, thereby attaining the second, expanded state of the prosthetic disc device 10 shown in FIG. 6B.

Although the description uses exemplary prosthetic disc device 10 to illustrate an exemplary implantation and deployment, persons of ordinary skill in the art will appreciate that this description may also apply to the prosthetic disc devices 50, 90 described above or any other variations thereof.

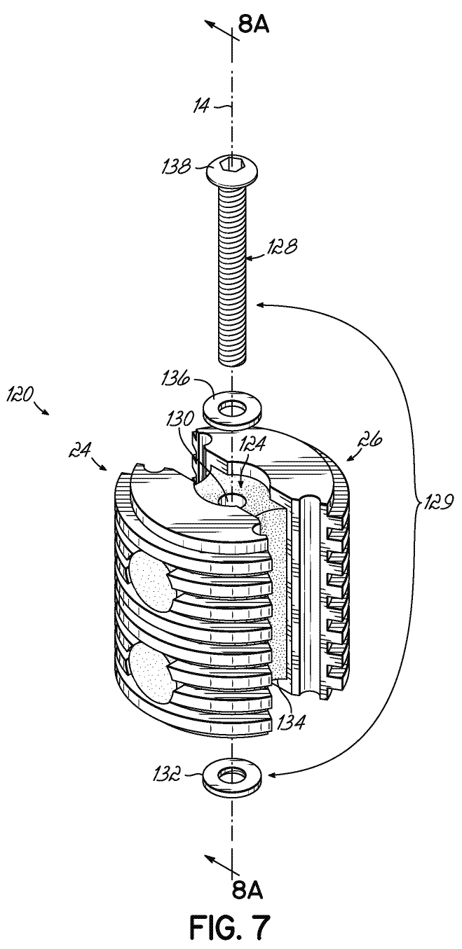

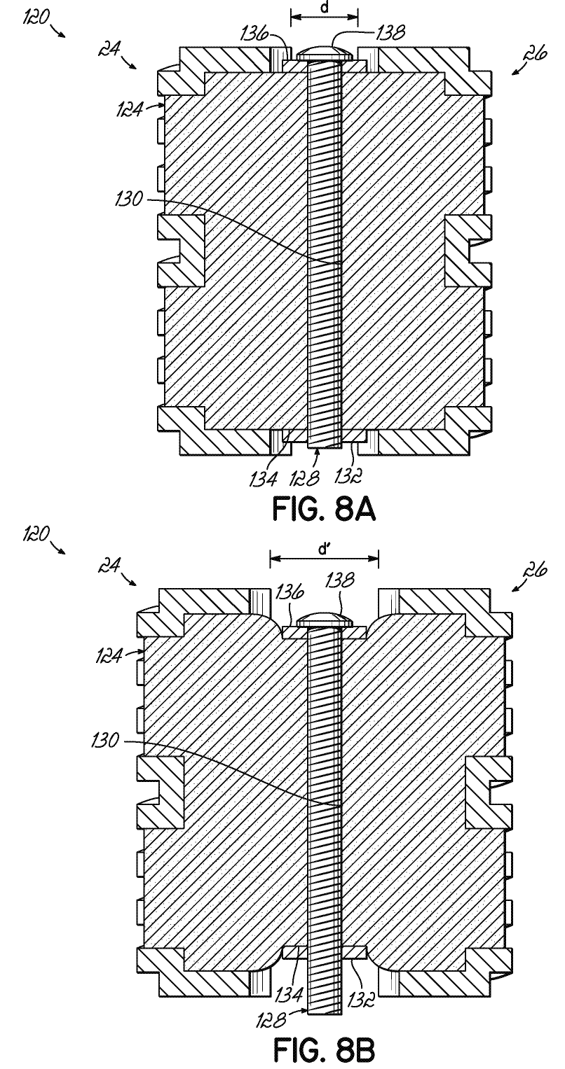

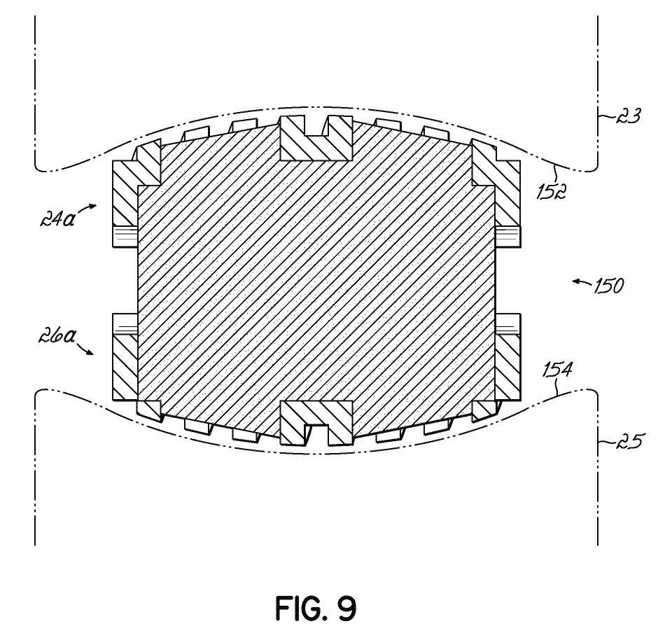

With reference to FIGS. 7, 8A-8B, in which like reference numerals refer to like features in FIGS. 1-2, there is shown an exemplary embodiment of a prosthetic disc device 120 similar in most respects to the prosthetic device 10, the description of which can be referred to for an understanding of prosthetic disc device 120 as well.

Prosthetic disc device 120 includes a core element 124 that is compressible in the direction of the central axis 14. The core element 124 includes an actuator 129 configured to collapse the core element 124 in such direction. The actuator 129 in the illustrative embodiment of FIG. 7 includes an aperture 130 adapted to receive a elongate threaded member such as a threaded bolt 128 therethrough as well a fastener engageable with a suitably chosen portion of the core element 124 to permit engagement of the fastener therewith.

In the embodiment of FIGS. 7, 8A-8B, the fastener is in the form of a threaded washer 132 adapted to receive the exemplary threaded bolt 128 and suspended in or engageable with a bottom face 134 of the core element 124. Persons of ordinary skill in the art will readily appreciate that the fastener may take any suitable form such as, and without limitation, a conventional threaded nut. Further, the fastener may lie anywhere within the core element 124.

Prosthetic disc device 120 may further include a bolt head washer 136 adapted to distribute the force applied by a head 138 of the bolt 128. Alternatively, a threaded washer or the like may be substituted for the conventional bolt head washer 136 and define the fastener described above.

With reference to FIGS. 8A-8B, the function of the actuator 129 of FIG. 7 is depicted. The prosthetic disc device 120 is implanted in an intervertebral bore 32 (FIG. 6A) such that the core element 124 is in a uncompressed state along the direction of the central axis 14, as shown in FIG. 8A (though it may be compressed in a direction orthogonal to the central axis 14, as described with reference to the embodiments of FIGS. 1-6B). Rotation of the threaded bolt 128 with respect to the threaded washer 132 causes compression of the core element 124, as shown in FIG. 8B, the deformation of which, due to a Poisson effect, results in an increase of a first spacing “d” between the first and second shells 24, 26 to a second spacing d′ (FIG. 8B).

With continued reference to FIGS. 8A-8B, this illustrative embodiment permits implantation of the prosthetic disc device 120 in an intervertebral bore 32 (FIGS. 6A-6B) and a post-implantation adjustment of the resulting intervertebral height (distance between adjacent vertebrae 23, 25).

While the embodiment of FIGS. 7, 8A-8B depict an actuator 129 in the form of an elongate threaded member and a fastener threadably engageable therewith, alternative configurations are contemplated including or not including the members described above, so long as the actuator 129 can be engaged by a user to cause compression of the core element 124 in a direction generally parallel to the central axis 14 to thereby cause an increase in the spacing between the first and second shells 24, 26. Such alternative configurations must such that compression of the core element 124 results in an increase of the intervertebral disc height defined by the implanted prosthetic disc device 120.

Similarly, while the actuator 129 is depicted as including one elongate threaded member and one fastener in cooperating relationship, persons of ordinary skill in the art will appreciate that actuator 129 may include members of the type described above in any number in excess of one.

With reference to FIG. 9, in which like reference numerals refer to like features in FIGS. 1-2 and 6A-6B, an alternative embodiment of a prosthetic disc device 150 is similar in most respects to the prosthetic disc device 10 (FIGS. 1-2), the description of which may be referred to for an understanding of prosthetic disc device 150 as well. Prosthetic disc device 150 includes first and second shells 24a, 26a, shaped to conform to the opposed surfaces 152, 154 of adjacent vertebrae 23, 25, respectively. The illustrative prosthetic disc device 150 may be implanted, as described above with reference to the embodiments of FIGS. 1-6B, by first defining an intervertebral bore 32 (FIG. 6A), or, alternatively and advantageously, without executing such step. In another advantageous aspect of this embodiment, the conformation of the first and second shells 24a, 26a may provide a secure fit of the prosthetic disc device 150 within the intervertebral space between vertebrae 23, 25.

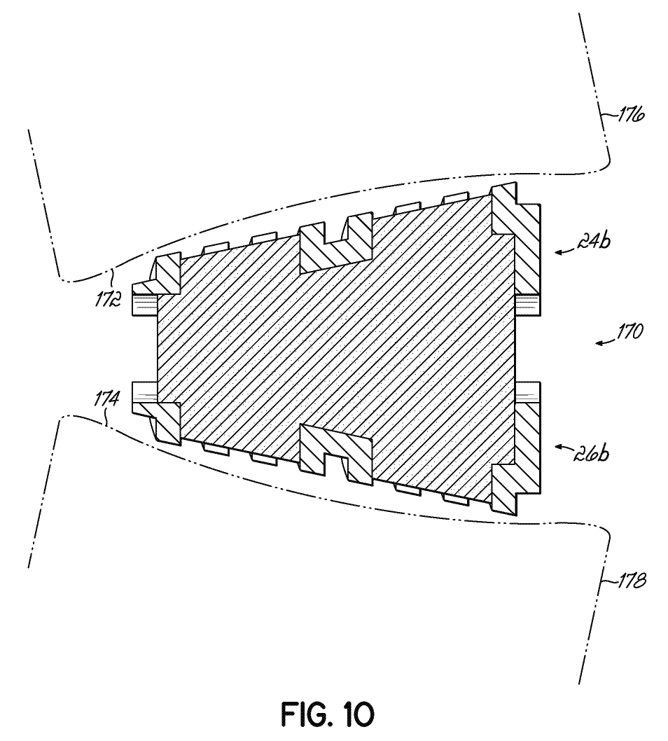

With reference to FIG. 10, in which like reference numerals refer to like features in FIG. 9, an alternative embodiment of a prosthetic disc device 170 is similar in most respects to the prosthetic disc device 150 (FIG. 9), the description of which may be referred to for an understanding of prosthetic disc device 170 as well. Prosthetic disc device 170 includes first and second shells 24b, 26b, shaped to conform to opposed surfaces 172, 174 of adjacent vertebrae 176, 178, respectively, in a spine segment where the adjacent vertebrae 176, 178 are angularly oriented to one another. For example, and without limitation, the prosthetic disc device 170 may be configured to conform to the opposed surfaces 172, 174 of adjacent vertebrae 176, 178 in a lordotic segment of a spine.

Advantageously, and due to their relatively small size as well as their shape, the prosthetic disc devices described above can be posteriorly implanted, thereby requiring less intrusive surgical procedures than those required for other known devices similarly seeking to restore disc height while preserving some of the natural relative motion between vertebrae.

While the present invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. The invention in its broader aspects is therefore not limited to the specific details, representative apparatus and method, and illustrative example shown and described. Accordingly, departures may be made from such details without departing from the spirit or scope of applicants' general inventive concept.

What is claimed is:

1. An implant for insertion between two adjacent vertebrae comprising:
an implant body having a leading end and a trailing end spaced apart by a longitudinal dimension of said implant;
two diametrically opposed first and second shells, each of said shells having a body portion and a thread portion extending outwardly from said body portion; and
a resilient support portion disposed between said two shells.

2. The implant of claim 1 wherein said support portion comprises an elastomeric material.

3. The implant of claim 1 wherein said support portion comprises a spring mechanism.

4. The implant of claim 1 wherein said support portion mechanically engages said two halves.

5. The implant of claim 1 wherein said support portion is adapted to collapse in a direction generally orthogonal to said longitudinal dimension thereby permitting said two shells to jointly define a threaded cylindrical body.

6. The implant of claim 1 wherein said shells and said support portion are integrally formed.

7. The implant of claim 1 wherein said thread portion is adapted to receive ingrown bone from one of the vertebrae.

8. The implant of claim 1 wherein said support portion is adapted to maintain said shells spaced apart.

9. The implant of claim 1 wherein said shells comprise a metal.

10. The implant of claim 1 wherein said thread portion extends substantially between said ends.

11. The implant of claim 5 wherein said threaded cylindrical body comprises a generally continuous threaded surface adapted to threadably engage two adjacent vertebrae.

12. The implant of claim 1 wherein said first and second shells are adapted to conform to respective shapes of opposed surfaces of adjacent vertebrae.

13. The implant of claim 12 wherein the adjacent vertebrae are oriented at an angle with respect to one another.

14. The implant of claim 13 wherein the adjacent vertebrae define a lordotic spine segment.

15. The implant of claim 1 wherein said support portion is further adapted to collapse in a direction generally parallel to said longitudinal dimension to thereby increase a spacing between said first and second shells.

16. The implant of claim 15 further comprising an actuator configured to collapse said support portion in said direction.

17. The implant of claim 16 wherein said actuator comprises at least one elongate threaded member and at least one fastener configured to threadably receive said elongate member.

18. An implant for insertion between two adjacent vertebrae comprising:
an implant body having a leading end and a trailing end spaced apart by a longitudinal dimension of said implant;
two diametrically opposed metallic first and second shells, each of said shells comprising a body portion and a thread portion extending outwardly from said body portion; and
an elastomeric resilient support portion disposed between said two shells and adapted to maintain said two shells spaced apart.

19. The implant of claim 18 wherein said support portion is further adapted to collapse in a direction generally orthogonal to said longitudinal dimension thereby permitting said two shells to jointly define a threaded cylindrical body.

20. The implant of claim 18 wherein said support portion comprises a spring mechanism.

21. The implant of claim 18 wherein said thread portion extends substantially between said ends.

22. The implant of claim 19 wherein said threaded cylindrical body comprises a generally continuous threaded surface adapted to threadably engage two adjacent vertebrae.

23. The implant of claim 18 wherein said first and second shells are adapted to conform to respective shapes of opposed surfaces of adjacent vertebrae.

24. The implant of claim 23 wherein the adjacent vertebrae are oriented at an angle with respect to one another.

25. The implant of claim 24 wherein the adjacent vertebrae define a lordotic spine segment.

26. The implant of claim 18 wherein said support portion is further adapted to collapse in a direction generally parallel to said longitudinal dimension to thereby increase a spacing between said first and second shells.

27. The implant of claim 18 further comprising an actuator configured to collapse said support portion in said direction.

28. The implant of claim 27 wherein said actuator comprises at least one elongate threaded member and at least one fastener configured to threadably receive said elongate member.

29. An implant for insertion between two adjacent vertebrae comprising:
an implant body having a leading end and a trailing end spaced apart by a longitudinal dimension of said implant;
two diametrically opposed first and second shells, each of said shells having a body portion and a thread portion extending outwardly from said body portion; and
a resilient support portion integrally formed with and disposed between said two shells and adapted to maintain said two shells spaced apart;
wherein:
said shells and said support portion are generally made from a material having at least two distinct regions, each of said regions respectively having first and second sets of physical properties;
at least a portion of said shells being made from material of said first region; and
said support portion being made from material of said second region.

30. A method of restoring an intervertebral disc height between two adjacent vertebrae, comprising:
posteriorly accessing a spinal column segment defined by the two adjacent vertebrae;
inserting at least one implant between the two vertebrae, the implant having two opposed body portions and a resilient support portion disposed between the two body portions.

31. The method of claim 30 further comprising defining an intervertebral bore between the two vertebrae prior to inserting the at least one implant therein.

32. The method of claim 30 further comprising moving the two body portions toward each other prior to inserting the at least one implant.

33. The method of claim 30 further comprising threadably engaging the implant with each of two confronting vertebral surfaces defining the intervertebral disc height.

34. The method of claim 33 further comprising threadably engaging the implant with cortical rims corresponding to each of the two vertebral surfaces.

35. The method of claim 30 further comprising inserting two implants between the two vertebrae.
